(19) United States
(12) Patent Application Publication
(10) Pub. No.: US 2009/0216072 A1
(43) Pub. Date: Aug. 27, 2009

(54) Urinary incontinence sling apparatus and method

(76) Inventor: Ralph Zipper, Melbourne, FL (US)

Correspondence Address: HAYWORTH, CHANEY & THOMAS, P.A., 202 N. HARBOR CITY BLVD., SUITE 300, MELBOURNE, FL, 32935, US

(21) Appl. No.: 12/072,648

(22) Filed: Feb. 27, 2008

(51) Int. Cl. A61F 2/02

(52) U.S. Cl. 600/30; 600/37

(57) ABSTRACT

A sling apparatus and method of treatment for stress urinary incontinence, prolapse, and the like. The apparatus and method may comprise a support material of any construction known within the art which is temporarily held in position by at least one tension member. A tension member may be attached to an end of the support material and the remaining free end of the tension member may then be releasably fastened to a fixation device at or above the level of the skin. Both the tension member and the fixation device may have incremental and/or measurable indicia disposed thereon to provide for either incremental increases or decreases in support material tension post-surgery. Upon support material natural fixation, the tension member may be cut at or below the level of the patient's skin. The tension member may comprise either permanent or bio-absorbable material.

### CROSS REFERENCE TO RELATED APPLICATIONS

Not applicable.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

1. Field of the Invention

The present invention generally relates to medical devices and methods, more specifically, the present invention relates to an adjustable urinary incontinence sling apparatus and method.

2. Background Art

Urinary incontinence, or the inability to control urination, is a major and debilitating problem affecting millions of people, especially women. The female's natural support system for the urethra is a hammock-like supportive layer composed of endopelvic fascia, the anterior vaginal wall, and a distal attachment to the pubic bone. Weakening and elongation of the pubourethral ligaments and the arcus tendineus fascia pelvis, weakening of the endopelvic fascia, and pubourethral prolapse of the anterior vaginal wall are some common characteristics of a patient with urinary incontinence.

Many procedures have been devised to treat urinary incontinence. Some have the goal of elevating the neck of the bladder to return it to a higher retropubic position. Many pubovaginal sling procedures have been developed to treat urinary incontinence. Some of these procedures involve positioning sling material under the urethra to provide elevation and support of the mid-urethra and/or the bladder neck. Examples of attachment sites for the sling include the anterior or superior portion of the pubis (e.g. with bone anchors and associated sutures), Cooper's ligament, or rectus abdominus fascia. Examples of procedures for treating incontinence are disclosed in U.S. Pat. Nos. 5,112,344; 5,611,515; 5,842,478; 5,860,425; 5,899,909; 6,039,686; 6,042,534; and 6,110,101.

Slings used for pubovaginal procedures may differ in the type of implantable material and anchoring methods used. In some cases, the sling is placed under the bladder neck and secured via suspension sutures to a point of attachment (e.g. bone) through an abdominal and/or vaginal incision.

The pubovaginal sling has gained widespread acceptance in the surgical management of stress urinary incontinence. The surgical procedure has undergone several modifications in an attempt to improve clinical outcomes including modifying the sling material to include, in whole or in part, synthetic, homologous, autologous, or porcine materials; altering the location of the suspension anchor among suprapubic, retropubic, and bone locations; and modifying the surgical position of the sling.

It is apparent that a very delicate balance exists between urinary incontinence and retention regardless of the sling material employed or the location of the sling suspension. Indeed, the primary factor to predict clinical success is related to the sling tension at the mid-urethra/bladder neck/sphincteric mechanism. If the tension of the pubovaginal sling is too loose, incontinence persists. If the sling is too tight at the bladder neck, urinary retention will develop. Previous attempts to regulate sling tension have not proven successful and the recommendation for sling tension is for surgeons to utilize “clinical judgment”. However, once the surgeon sets the tension during surgery, the tension cannot be adjusted after the surgery is completed.

In addition, recent data suggests that after an extended period of time, the suspension suture is indeed redundant because of the perivesical fibrosis that anchors the bladder into its fixed, high retropubic position. Moreover, the sling itself serves as a matrix for fibroblast deposition, which strengthens and supports the anterior vaginal wall.

Complications associated with procedures for treating incontinence include urinary retention, bladder instability and erosion of an implanted article into surrounding tissue. See Spencer et al, A Comparison of Endoscopic Suspension of the Vesical Neck With Suprapubic Vesicourethropexy for Treatment of Stress Urinary Incontinence, J. Urol. 137: 411, (1987); Araki et al, The Loop Loosening Procedure for Urination Difficulties After Stamey Suspension of the Vesical Neck, J. Urol., 144; (1990); and Webster et al., Voiding Dysfunction Following Cystourethropexy: Its Evaluation and Management, J. Urol., 144 (1990).

With respect to sling procedures, if the sling mesh is too loosely associated with its intended physiological environment, the mesh may be ineffective in supporting the urethra and treating incontinence. Several complications can arise from mesh that is too tightly placed and such complications may include retention, sling erosion, and other damage to surrounding tissue such as the urethra and vagina.

Proper tension of a sling during and after the surgical intervention is an important factor for successful treatment of incontinence. Surgical approaches to applying tension or slack in a sling procedure vary widely. See Decter, Use of the Fascial Sling for Neurogenic Incontinence: Lessons Learned, The Journal of Urology, Vol. 150, 683-686 (1993).

One example of an adjustable sling device may be found in U.S. Pat. No. 6,881,184 issued to Zappala. The reference, however, discloses an absorbable pubovaginal sling system comprising an absorbable sling and a looped monofilament suture that is adapted to be transposed to the suprapubic position, supported by an external adjustable tension device, and connected to the absorbable sling. The reference exclusively discloses suprapubic use of an absorbable sling and permanent looped sutures to treat only urinary incontinence. The present invention discloses the preferable use of a permanent sling with absorbable sutures to adjustably treat a variety of disorders including urinary incontinence, fecal incontinence, prolapse, and the like via possible device placement within a variety of previously unused bodily spaces. The present invention improves upon and serves to correct and/or eliminate pitfalls which commonly occur when adhering to the Zappala disclosure and structures. Such pitfalls may include higher failure rates, increased infections, and sinus tract formation.

Other prior art sling procedures use bone anchors or other methods for securing a sling. Some attachment sites, such as the rectus abdominus fascia or the top of the pubic bone, require very long sutures that contribute to the unnatural positioning of the urethra. Long sutures increase the difficulty in achieving the proper tension in the sutures and sling and increase the chances that intervening anatomical structures may interfere with proper tension. Improper sling tension or sling suture tension can result in increased lateral movement and momentum of the support structures or mesh sling when they are moved due to intra-abdominal pressures.

Because many slings are anchored at anatomical positions remote from the urethra, proper tension in a sling is a difficult objective to achieve. Results can vary widely. The present invention provides a solution to this difficult objective.

### BRIEF SUMMARY OF THE INVENTION

Because many slings are anchored at anatomical positions remote from the urethra, proper tension in a sling is a difficult objective to achieve. Results can vary widely. The Absent invention provides a solution to this difficult objective.

### BRIEF DESCRIPTION OF THE DRAWINGS

FIG. 1A depicts a perspective view of an embodiment of the present invention immediately following completion of the inventive method wherein one support material B end is disposed in the subcutaneous space H and a second support material B end is disposed in the periurethral, anterior vaginal, and/or perineal space G.

FIG. 1B depicts a perspective view of an embodiment of a final result of the present invention having one support material B end disposed in the subcutaneous space H and a second support material B end disposed in the periurethral, anterior vaginal, and/or perineal space G.

FIG. 1C depicts a perspective view of another embodiment of a final result of the present invention having one support material B end disposed in the subcutaneous space H and a second support material B end disposed in the periurethral, anterior vaginal, and/or perineal space G.

FIG. 2A depicts a side cross sectional view of an embodiment of the present invention immediately following completion of the inventive method wherein the ends of the support material B may be disposed in the suprapubic space K.

FIG. 2B depicts a side cross sectional view of an embodiment of a final result of the present invention having the ends of the support material B disposed in the suprapubic space K.

FIG. 2C depicts a side cross sectional view of another embodiment of a final result of the present invention having the ends of the support material B disposed in the suprapubic space K.

FIG. 3A depicts a side cross sectional view of an embodiment of the present invention immediately following completion of the inventive method wherein the ends of the support material B may be disposed in the retropubic space I.

FIG. 3B depicts a side cross sectional view of an embodiment of a final result of the present invention having the ends of the support material B disposed in the retropubic space I.

FIG. 3C depicts a side cross sectional view of another embodiment of a final result of the present invention having the ends of the support material B disposed in the retropubic space I.

FIG. 4 depicts a perspective view of bodily spaces in regards to the support material B positioning of embodiments of the present invention.

FIG. 5 depicts a side cross sectional view of bodily spaces in regards to the support material B positioning of embodiments of the present invention.

FIG. 6 depicts an embodiment of the present invention incorporating marks or indicia disposed on the tension member A and/or on the fixation device F used for retention thereby allowing for incremental increases or decreases in support material B tension.

### FIGURE REFERENCE NUMERALS

A Tension member
B Support material
C Obturator membrane of subject patient
D Bladder of subject patient
E Urethra of subject patient
F Fixation device on or above skin of subject patient
G Periurethral, anterior vaginal, and/or perineal spaces
H Subcutaneous spaces
I Retropubic space
J Prepubic space
K Suprapubic space
R Rectus muscle of subject patient
V Vagina of subject patient
X Rectus fascia of subject patient

### DETAILED DESCRIPTION OF THE INVENTION

Stress urinary incontinence is broadly defined as the involuntary loss of urine with activities that cause an increase in intraabdominal pressure. Examples of such activities include coughing, laughing, sneezing, bending, yelling and exercising. Sling procedures comprise a broad class of surgical procedures which may be used to treat stress urinary incontinence. Conventional sling procedures may involve the placement of natural or synthetic material or the patient's own tissue under, around, or near the urethra. The support material disposed about the urethra for treating urinary incontinence is commonly referred to as a sling. When appropriately placed and without subsequent movement following the procedure, the support material provides a cure for a patient's stress urinary incontinence.

All existing methods of support material placement, such as sling procedures, are associated with both high failure and complication rates. Failures are typically caused by two factors. The first factor involves the support material either not being placed tightly enough around or close enough to the tissue to be supported, such as the urethra. The second factor involves the support material being initially placed correctly by the surgeon, but thereafter the support material moves into an inappropriate position. Such support material movement may be caused by an increase in intraabdominal pressure before the support material has had a chance to become held in a fixed position by scar tissue formation.

Sling procedures and similar applications utilizing support material may typically involve two types of complications. The first type of complication may be urinary retention. Many patients have difficulty urinating and such a condition may be permanent. This complication may occur when the support material is placed too tightly or too close to the urethra. Treatment, when possible, requires surgical removal, cutting, or loosening of the support material. The second type of complication may be bladder injury. Bladder injury may occur when the support material is inadvertently placed inside the bladder, bladder wall, or bladder muscle. Support material left inside the bladder may lead to severe infection and cause an overactive bladder. Treatment typically requires a major surgery. Support material left inside the bladder wall or bladder muscle may often lead to pain and a severely overactive bladder. This treatment involves a major surgery and is often unsuccessful.

The present invention discloses both a system and method for substantially reducing and potentially eliminating the failures and major complications of sling procedures and all similar medical procedures known within the art. The present invention may conveniently be incorporated into all known sling techniques and may be undertaken using conventional slings and surgical tools. In accordance with the present invention, tension adjustments to the implanted support material may be made without any need to enter the vagina and/or make an incision.

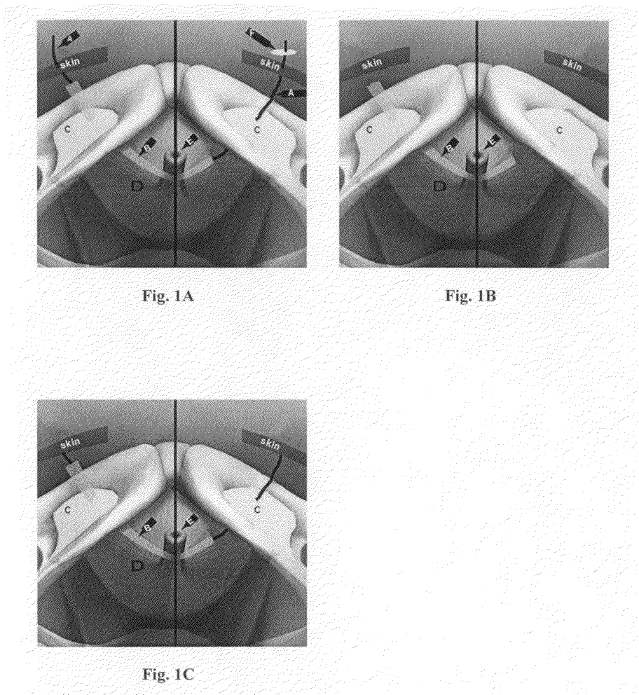

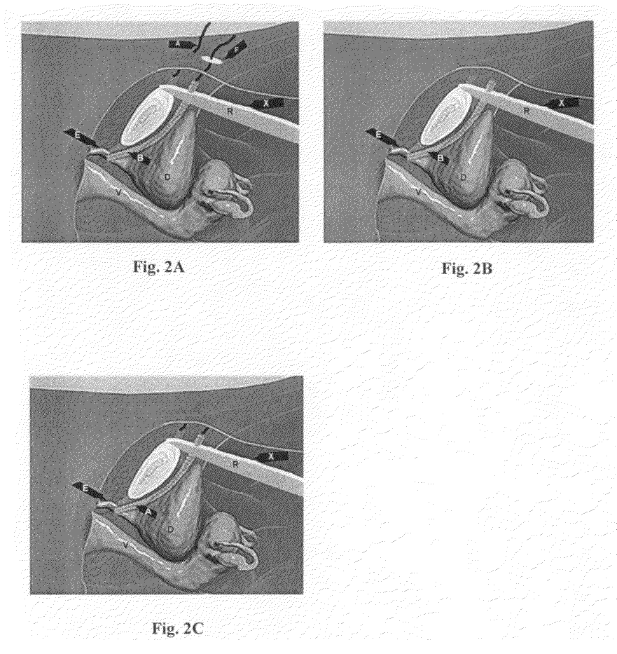

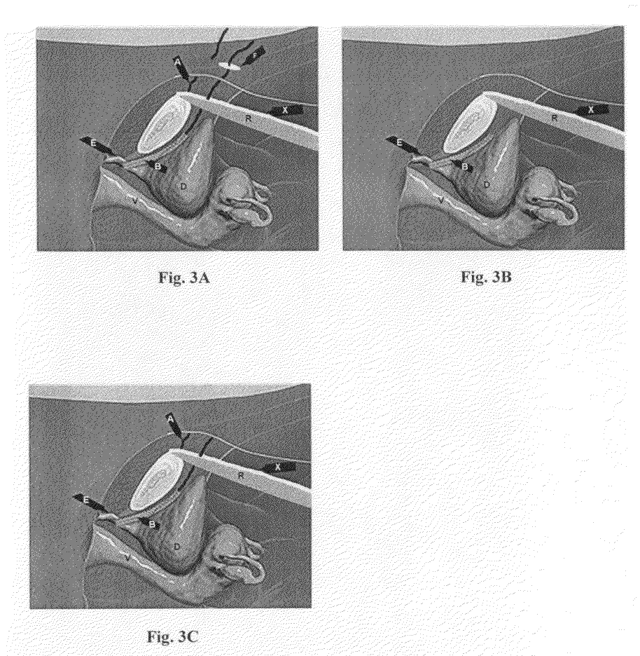

As depicted in FIGS. 1-6, the present invention may comprise at least one tension member A, such as a suture or other device, being secured at or above the level of the skin as a means for allowing tension adjustment of a support material B disposed with a patient. The at least one tension member A may preferably attach to a terminal end of the support material B, wherein the support material B may not require a sheath for placement. The tension member A may be comprised of permanent or bio-absorbable material. The tension member A may also be releasably attached to a fixation device F at or above the skin level of the patient (see FIGS. 1A, 2A, 3A, and 4-6). The fixation device F may release tension member A and allow for tension adjustments via manipulation of the proximal terminal end of the tension member A. Such tension adjustments may serve to either increase or decrease tension with minimal deformity to the support material B architecture, wherein the tension member A is again retained by the fixation device F post-adjustment.

After a short period of time, which generally may be no less than 24 hours and no more than 6 weeks, the tension member A may then be cut at or below the level of the skin (see FIGS. 1C, 2C, and 3C). Prior to cutting, the tension member A prevents movement of the support material B during increases in intraabdominal pressure. The longer the tension member A is left in place, the better the chance that the support material B will be sufficiently scarred in place and be able to resist increases in intraabdominal pressure and movement. When the tension member A is cut after scarring is completed, surgical failure due to support material B movement is unlikely. Surgical failure may still occur if the support material B it is not placed tightly enough or close enough to the tissue to be supported (e.g. the urethra E).

Within the scope of the present invention, the at least one tension member A may comprise any means of attachment to the support material B that is known within the art including but not limited to sutures, stitches, lines, threads, filaments, fibers, strings, cords, ropes, cables, staples, and the like. Similarly, the at least one fixation device F providing for retention of the tension member A at or above the level of the skin may comprise all means of fixation known within the art include but not limited to an interface with the skin itself, bandages, gauze, casts, dressing, swathes, wrappings, bindings, fasteners, closures, couplings, buttons, and the like.

The scope of the present invention includes use of the disclosed system and method in a wide variety of medical procedures including but not limited to treatments for urinary incontinence, fecal incontinence, vaginal prolapse, and the like. While the description and figures may primarily pertain to treatment of urinary incontinence, this is merely an illustrative example and the scope of the present invention is useful in all similar medical procedures providing support.

The present inventive apparatus and method allow for adjustment of the support material B after surgery has been completed. Any time after the surgery, the surgeon or other technician may release a tension member A from its associated fixation device F at or above the skin while incrementally increasing the tension on the support material B. This incremental increase in tension may be measured by marks or indicia disposed on the tension member A and/or on the fixation device F used for retaining the tension member A (FIG. 6). Following the application of increased tension, the surgeon or other technician may again refasten the tension member A to the fixation device F at or above the skin.

Urinary retention, difficulty with urination or urine stream may be caused by an over tensioning of the support material B or placement of surgical support material too close to the urethra E. The present invention may be used to correct such post-surgery complications. The surgeon or other technician may, anytime after the surgery but before the final cutting of a tension member A, release the tension member A from its fixation device F and incrementally decrease the tension applied to the support material B.

This incremental decrease in tension may be measured by marks or indicia on the tension member A and/or the fixation device F used for retaining the tension member A (FIG. 6). Following the decrease in tension, the surgeon or other technician may again refasten the tension member A to the fixation device F at or above the skin.

The present inventive method may be applied to any type of support material B known within the art. The method may further allow surgeons to choose shorter support materials B which may result in a lower incidence of support materials B being left within the bladder D, the bladder wall, or the bladder muscle.

One preferred embodiment of the present invention, generally depicted in FIG. 1A, comprises two fixation devices F, a permanent support material B, and two absorbable tension members A. Each respective end of the permanent support material B may have a tension member A attached thereto. Each of the two respective tension members A may then make their way to the skin level of the patient and be releasably fastened to each respective fixation device F. When the present invention is employed using an absorbable tension members A, the support material B will eventually remain in the patient's body as an isolated implant after tension member A absorption. All known absorbable materials, including but not limited to polyglactin and materials having similar absorption time tables, may compose the absorbable tension members A of the present invention. Shortcomings of permanent tension members A include a high incidence and recurrence of infection as well as sinus tract formation. Use of an absorbable support material B may also prove detrimental as fibroblast migration and collagen deposition may be too slow and insufficient to provide a durable cure for many patients.

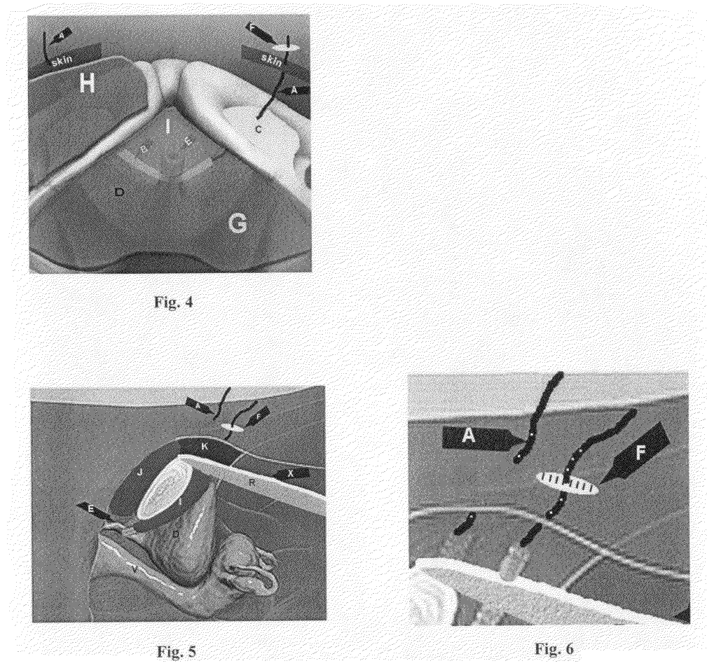

The present inventive method and apparatus comprising a support material B disposed within a patient's body may further comprise at least one tension member A that may travel beyond any body space or cavity G, H, I, J, K in order to reach the skin level of the patient (see FIGS. 4 and 5). However, once the tension member A is absorbed, an extension beyond any cavity or space G, H, I, J, K in which the support material B resides no longer exists (see FIGS. 1A, 2A, and 3A, pre-absorption; FIGS. 1B, 2B, and 3B, post-absorption). Such an absorption of the tension member A may serve to decrease any risk of infection or communication between the support material B and the patient's skin. The composition of the tension member A is selected such that it remains unabsorbed for a sufficient period of time to allow early adjustment of and subsequent fixation via scarring of the support material B.

Another embodiment of the present invention may comprise at least one temporary fixation device F having at least one permanent tension member A. When the present invention is employed using a permanent tension member A, all the benefits of adjustment and fixation, as described above, remain. However, as the tension member A remains permanently in position and attached to the support material B after the tension member A may be cut at or below the skin level (see FIGS. 1C, 2C, and 3C), the tension member A may provide a continuing resistance against support material B movement and thereby further decrease support material B failure rates. However, any decreased risk of infection associated with an absorbable tension member A may not be realized when using a permanent tension member A. Furthermore, there will be a permanent tension member A extension from the support material B, wherein some embodiments may comprise tension member A extensions that remain below skin levels and other embodiments may comprise tension member A extensions that extend beyond the space or cavity G, H, I, J, K in which the support material B resides.

Having at least one temporary fixation device F disposed at or above the level of the skin, the present inventive disclosure is beneficial and useful for any manner of medical procedure providing support or tension on a tissue or device and may further be utilized on all areas of the body including but not limited to urological and gynecological regions. Regarding such pelvic regions specifically, the present invention allows for treatment of incontinences, prolapse, and the like via use of conventionally known body regions as well as use of novel regions for such treatments. The present inventive disclosure allows for treatment using the pubovaginal, transobturator, prepubic, retropubic, suprapubic, rectus muscle, and rectus fascia regions as well as the subcutaneous, periurethral, anterior vaginal, perineal, supraobturator, and preprepubic spaces.

While the above description contains much specificity, these should not be construed as limitations on the scope of any embodiment, but as exemplifications of the presently preferred embodiments thereof. Many other ramifications and variations are possible within the teachings of the various embodiments.

Thus the scope of the invention should be determined by the appended claims and their legal equivalents, and not by the examples given.

1. A system for holding a support material in place following a medical procedure, comprising:
said support material having a first end and a second end;
at least one tension member, wherein said at least one tension member attaches to said first end or said second end of said support material; and
at least one fixation device, wherein said at least one fixation device releasably retains said at least one tension member, said at least one fixation device being disposed at or above the level of the skin and allowing for tension adjustment of said at least one tension member.

2. The system of claim 1, wherein said medical procedure is selected from the group consisting of urinary incontinence treatment, fecal incontinence treatment, and vaginal prolapse treatment.

3. The system of claim 1, wherein said at least one tension members comprise absorbable material.

4. The system of claim 1, wherein said support material comprises a permanent, non-absorbable material.

5. The system of claim 1, wherein said at least one fixation devices and/or said at least one tension members further comprises marks or indicia disposed on to assist in said tension adjustment.

6. The system of claim 1, wherein said first end and said second end of said support material terminate in a body space selected from the group consisting of the pubovaginal space, the transobturator space, the prepubic space, the retropubic space, the suprapubic space, the rectus muscle, the rectus fascia, the subcutaneous space, the periurethral space, the anterior vaginal space, the perineal space, the supraobturator space, and the preprepubic space.

7. The system of claim 1, wherein at least said first end and/or said second end of said support material terminates in a body space selected from the group consisting of the periurethral space, the anterior vaginal space, the perineal space, the supraobturator space, and the preprepubic space.

8. The system of claim 1, further comprising:
at least two tension members, wherein at least a first tension member is attached to said first end of said support material and at least a second tension member is attached to said second end of said support member; and
at least two fixation devices, wherein a first fixation device releasably retains said first tension member and a second fixation device releasably retains said second tension member, said at least two fixation devices being disposed at or above the level of the skin and allowing for tension adjustment of said at least two tension members.

9. A method of holding a support material in place following a medical procedure, comprising the steps of:
providing said support material having a first end and a second end;
providing at least one tension member, wherein said at least one tension member attaches to said first end or said second end of said support material;
providing at least one fixation device, said at least one fixation device being disposed at or above the level of the skin and allowing for tension adjustment of said at least one tension member;
passing said support member beneath a tissue to be supported;
transposing said at least one tension member to a level at or above the level of a patient's skin;
passing said at least one tension member through said at least one fixation device;
adjusting tension of said at least one tension member to a desired level; and
releasably retaining said at least one tension member with said at least one fixation device.

10. The method of claim 9, wherein said medical procedure is selected from the group consisting of urinary incontinence treatment, fecal incontinence treatment, and vaginal prolapse treatment.

11. The method of claim 9, wherein said at least one tension members comprise absorbable material.

12. The method of claim 9, wherein said support material comprises a permanent, non-absorbable material.

13. The method of claim 9, wherein said at least one fixation devices and/or said at least one tension members further comprise marks or indicia disposed thereon to assist in said tension adjustment at or above the level of the skin.

14. The method of claim 9, wherein said first end and said second end of said support material terminate in a body space selected from the group consisting of the pubovaginal space, the transobturator space, the prepubic space, the retropubic space, the suprapubic space, the rectus muscle, the rectus fascia, the subcutaneous space, the periurethral space, the anterior vaginal space, the perineal space, the supraobturator space, and the preprepubic space.

15. The method of claim 9, wherein at least said first end and/or said second end of said support material terminates in a body space selected from the group consisting of the periurethral space, the anterior vaginal space, the perineal space, the supraobturator space, and the preprepubic space.

16. The method of claim 9, further comprising:
providing at least two tension members, wherein at least a first tension member is attached to said first end of said support material and at least a second tension member is attached to said second end of said support member; and
providing at least two fixation devices, wherein a first fixation device releasably retains said first tension member and a second fixation device releasably retains said second tension member, said at least two fixation devices being disposed at or above the level of the skin and allowing for tension adjustment of said at least two tension members.

17. The method of claim 9, further comprising:
adjusting said tension of said support material following completion of said medical procedure without the need to close an incision.

18. The method of claim 9, further comprising:
adjusting said tension of said support material at the level of the skin following completion of said medical procedure.

19. The method of claim 9, further comprising:
leaving no permanent material beyond that of said support material following said tension adjustment of said support material.

20. The method of claim 9, further comprising:
adjusting said support material at or above the level of the skin via at least one permanent extension from said support material.
